(19) United States
(12) Patent Application Publication
(10) Pub. No.: US 2004/0162533 A1
(43) Pub. Date: Aug. 19, 2004

(54) Applicator device

(76) Inventor: Kenneth A. Alley, Berwick, PA (US)

Correspondence Address: LAW OFFICES OF MARK A. GARZIA, P.C., 2058 CHICHESTER AVE, BOOTHWYN, PA, 19061, US

(21) Appl. No.: 10/773,786

(22) Filed: Feb. 6, 2004

Related U.S. Application Data
(60) Provisional application No. 60/445,256, filed on Feb. 6, 2003.

(51) Int. Cl. A61M 35/00

(52) U.S. Cl. 604/290

(57) ABSTRACT

A device for applying a substance, for example an antiseptic, to the skin of a person. The device includes a a container having an open end for storing a liquid; an applicator head that has a delivery means at one end and a piercing element at the opposite end; the applicator head communicates with said container for delivering the liquid to the delivery means; a barrier positioned proximate said open end of the container for preventing the liquid from escaping said container until such time as the cap is manipulated (e.g., twisted) with respect to the container thereby allowing the piercing element to penetrate or break the barrier.

### CROSS REFERENCE TO RELATED APPLICATIONS

The present application claims the benefits under 35 U.S.C. §119(e) of U.S. Provisional Application No. 60/445,256 filed Feb. 6, 2003, titled ALLEY APPLICATOR DEVICE in the name of Kenneth A. Alley.

U.S. Provisional Application No. 60/445,256 filed Feb. 6, 2003, is hereby incorporated by reference as if fully set forth herein.

### FIELD OF THE INVENTION

The present invention relates generally to applicator devices that are commonly used for the preparation of a patient's skin. More specifically, the present invention is an applicator device to be used for anti-microbial antiseptics that require a user-friendly, single-dose treatment.

### BACKGROUND OF THE INVENTION

There are a host of applicator devices on the market that are used for the purpose of preparing the patients skin prior to medical procedures. The most common skin prep procedure includes submerging a sponge or foam absorbent pad into an antiseptic solution and wiping the soaked foam pad over the patient's skin. The fingers (or glove) of the nurse or other care practitioner are allowed to touch the foam pad and the antiseptic solution. The solution may then be absorbed by the nurse's skin or have a detrimental effect on the material of the glove.

Other devices adapted for single-dose delivery systems incorporate a glass ampule filled with an aseptic solution. The glass ampule is then placed within a second, flexible container (preferably plastic) with the open end of the second container having a sponge or foam pad.

In order to activate these delivery systems, the clinician needs to forcefully flex the outer container with sufficient force to break the glass ampule. The contents of the glass ampule then pour out into the outer plastic container and, when titled in the appropriate fashion onto the foam pad. When the foam pad is sufficiently soaked through, the aseptic solution may be applied by wiping the pad over the patient's skin.

The use of glass ampule requires expensive and specialized production and filling equipment. In addition, broken glass poses other potential hazards to both the patients and the clinicians. Another problem with these existing devices is that the clinicians must use significant force to flex the device in order to break the glass ampule. This physical force directly on the outer container increases the risk of contaminating the foam pad prior to use. Also, the disposal of this delivery system is more of a problem because of the combination of plastic and glass, which cannot be easily separated.

Accordingly, there is a need for a safe, user-friendly and economical single-dose aseptic delivery system that does not require the breaking glass to activate the flow of the solution.

### SUMMARY OF THE INVENTION

The present invention is a self-contained, tamper-proof, pre-filled, single use application device for use with topical antiseptics for reducing skin bacteria and other harmful contaminants prior to a medical procedure. The present invention uses a standard container that consists of a unique bottleneck finish. The container is preferably cylindrical in shape to allow it to be easily manufactured and held during use. The container has an open end that is covered over by a foil liner, plastic plug or similar cap acceptable for sealing the end of the container.

The present invention also consists of an applicator head with a foam pad at one end and means to communicate with the unique bottleneck finish at the other end. The applicator head will lock and seal onto the unique bottleneck finish of the container; then, when the applicator head is rotated, it will activate the device by piercing the foil liner on the container. The liquid will flow out of the container, up into the applicator head and soak the foam pad.

The applicator head may include safety finger grips to reduce the liklihood of contaminating the foam pad during activation. These finger grips allow the clinician to more easily activate the present invention without touching the foam absorbent pad. The present invention includes aseptic means to activate the flow of antiseptics to the absorbent pad, which provides the means to frictionally apply the antiseptics to the skin.

Unlike other single use applicators, the present invention does not require the use of a glass ampule. Further, the present invention can be manufactured and pre-filled utilizing standard production and filling equipment.

### BRIEF DESCRIPTION OF THE DRAWINGS

The foregoing summary, as well as the following detailed description may be better understood when read in conjunction with the accompanying drawings, which are incorporated in and form a part of the specification. The drawings serve to explain the principles of the invention and illustrate embodiments of the present invention that are preferred at the time the application was filed. It should be understood however that the invention is not limited to the precise arrangements and instrumentalities shown.

FIG. 1 is a cut-away side view of an applicator device in accordance with the present invention.

### DETAILED DESCRIPTION OF THE PREFERRED EMBODIMENT

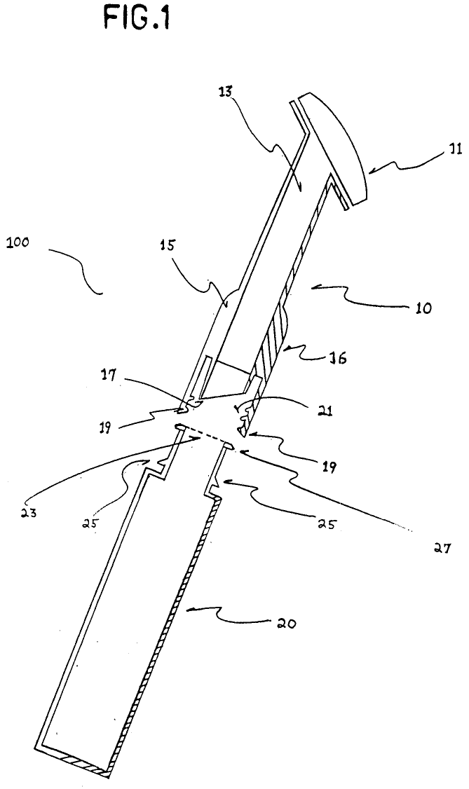

Referring now to FIG. 1, an applicator device in accordance with the present invention is indicated at 100. In a preferred embodiment, the applicator device is designed to deliver topical antiseptics for reducing skin bacteria and other harmful contaminants prior to a medical procedure (e.g., injecting the patient with a needle).

In a preferred embodiment, the apparatus 100 comprises two primary parts; namely, an applicator head 10 and a container 20.

The container 20 has an open end 12 that allows the container to be filled with a solution during manufacturing. In most cases, the solution will be a liquid aseptic, but could be a fine powder or any solution that can flow out of the container. Once the container is filled, the open end is sealed with a foil liner, plastic cap or other appropriate cover 23.

Proximate the open end 12 of the container are tapered flanges 27 and reverse undercut bottle locks 25. In a preferred embodiment, the tapered flanges are manufactured in the shape of an oval. The tapered flanges 27 and the bottle locks 25 form a locking and activation mechanism that will be described more fully hereinafter.

The applicator head 10 includes an elongated tubular section 13, a foam pad 11 (or other means for absorbing the solution on one side and passing it through to the other), a locking mechanism 19, an activation mechanism 21, and a means for piercing 17 the foil liner or cover 23 of the container 20.

The locking grips 19 are designed to communicate with bottle locks 25 of the container to secure the applicator head 10 to the container 20. The combination locking grips 19 and the bottle locks 25 form a locking mechanism that is designed to secure the applicator head 10 to the container 20. The activation mechanism 21 is designed to communicate with the tapered flanges 27 in order to allow the piercing means 17 to penetrate the foil liner 23, thereby providing an escape means for the liquid stored in the container 20.

In a preferred embodiment, the activation mechanism 21 are one or more threads that matingly engage the tapered flanges 27. Initially, the tapered threads 21 may work in conjunction with the locking mechanism 19, 25 to secure the applicator head 10 to the container 20; this is especially desirable when shipping the applicator device 100. If the tapered flanges are oval-shaped, an approximately 1/4 turn will provide sufficient movement of the applicator head 10 with respect to the container 20 to allow the piercing element 17 to penetrate the foil seal 23 while simultaneously continuing to ensure that the applicator head 10 remains firmly attached to the container 20.

The tapered threads 21 have a dual purpose; during assembly, the tapered threads 21 engage the flanges 27 thereby preventing the accidental penetration of the foil seal 23. The tapered threads 21 act as a physical stopping means until the clinician intentionally activates the applicator device 100 by rotating the applicator head 10 with respect to the container 20. During the rotation, the flanges 27, physically engage the threads 21 and draw the applicator head 10 down towards the container 20.

The activation of the applicator device 100 by rotating the applicator head 100 with respect to the container 20 does not affect the hermetic seal between the applicator head and container. The piercing element 17 does not need to make a large hole in the foil seal—only a relatively minor penetration will be sufficient to completely release the solution stored in the container. Once the solution has been released, it is free to travel through the tubular section 13, which forms a passageway directly from the container 20 to the foam pad 11. When the solution hits the foam pad 11, it is absorbed and the clinician may apply the solution to the skin of the patient.

The outer side of the applicator head 10 may include finger indents 15, 16 to allow the clinician to more easily grasp and rotate the applicator head 10. Instead of finger indents (or in addition to the finger indents), a rubberized coating having a specialized texture may be applied to a portion of the outside of the applicator head 10 to assist in holding/manipulating the applicator head.

Activating the apparatus 100 requires rotation of applicator container 20. Apparatus 100 is held snugly by safety finger grips 15 and 16 of applicator head 10 and applicator container 20 is rotated where the ovalized tapered flanges 27 of container 20 engage threads 21 of applicator head 10. The applicator head piercing edge 17 is forced through foil liner 23 thus, opening passageway 13 of applicator head 10. The locking means 19 engage and lock onto the reverse undercut bottle lock 25 preventing any further movement of applicator head 10 with respect to container 20. Once the foil liner is pierced, the solution is free to flow (via gravity) through passageway 13 and into absorbent foam head 11. Foam head 11 of apparatus 100 may be used to apply a friction application of the solution in container 20.

In a preferred embodiment, the cylindrical bottle will be filled with an antiseptic and sealed with a foil linier by heat induction. The applicator head 10 will then be snapped onto the container 20, which will lock the two components together. The applicator device may then be packaged and sterilized by e-beam or gamma radiation. When needed to activate the device, the clinician/operator holds the safety grips 15, 16 and rotates either the applicator head or the container approximately ¼ turn (or however number of turns necessary to pierce the foil, and to simultaneously ensure the integrity and sterility of the applicator device 100).

The applicator head will maintain an aseptic seal between the applicator head and the contents of the container. When the necessary number of turns needed to activate the device are made, the applicator head will penetrate the foil liner of the container and lock onto the bottleneck finish of the container. The solution will freely flow through the inner passageway of the applicator head and into the hydrophilic foam head. The applicator device can then be used to provide an effective friction rub of the patient's skin minimizing the potential of microbial contamination.

There are numerous anti-microbial agents and antiseptics that may be used in such as device, such as isopropyl alcohol, chlorhexidine gluconate, and iodine to name a few.

Besides medical applications, the applicator device 100 may be used for cosmetic, industrial and other non-medical applications as well. For example, the applicator device may deliver nail polish, foundation, paints, chemicals, adhesives, etc.

After reading this disclosure, a person skilled in the art may recognize other means for locking the applicator head 10 to the container 20, and manipulating the applicator head and/or container in order to penetrate the cap 23. For example, a plastic cap having a door attached via a living hinge on one side and scored to break free on the remaining three sides may replace the foil liner. By manipulating the applicator head 10, the scored portion of the door may break away, thereby opening the door and releasing the solution into the tubular section 13.

Although this invention has been described and illustrated by reference to specific embodiments, it will be apparent to those skilled in the art that various changes and modifications may be made which clearly fall within the scope of this invention. The present invention is intended to be protected broadly within the spirit and scope of the appended claims.

I claim:

1. A device for applying a substance, for example to the skin of a person, the device comprising:
a container having an open end and a closed end;
a barrier positioned proximate said open end of the container for preventing the substance from escaping said container;
an applicator head having a delivery means, said applicator head communicating with said container for removably attaching the applicator head to the open end of said container, said applicator head including a passageway for delivering the liquid from the container to the delivery means; and
means associated with said applicator head for opening said barrier after said applicator head is attached to the container by manipulating said applicator head with respect to the container.

2. The device of claim 1 wherein said delivery means is an absorbent pad.

3. The device of claim 2 wherein said absorbent pad is made of foam.

4. The device of claim 1 wherein said delivery means is a hydrophillic absorbent material.

5. The device of claim 1 wherein said delivery means is made of terry cloth.

6. The device of claim 1 wherein said barrier is a foil liner.

7. The device of claim 1 wherein said means for opening is a projection having a piercing element.

8. The device of claim 7 wherein said barrier is a plug able to be penetrated by said piercing element.

9. The device of claim 8 wherein said plug is made of a plastic material.

10. The device of claim 1 wherein said container is sized to deliver a single dosage of said substance.

11. The device of claim 1 wherein said substance is topical medicament for applying to a section of skin.

12. The device of claim 1 wherein said substance is topical antiseptic for sterilizing a section of skin.

13. The device of claim 12 wherein said topical antiseptic is alcohol.

14. The device of claim 1 wherein said container is pre-filled with a pre-measured substance.

15. The device of claim 1 wherein said device is sold and/or delivered in a tamper-proof packaging.

16. The device of claim 1 wherein said substance is a viscous solution.

17. The device of claim 1 wherein said substance is a powder.

18. An applicator device for dispensing a liquid substance, the device comprising:
a container for storing the liquid substance, the container having an open end for allowing the filling thereof with the liquid substance;
a barrier over said open end of the container for sealing in the liquid substance;
an applicator head having a cotton pad at a first end, a means for communicating with the open end of the container at a second end, and a tubular section for connecting the pad to the open end of the container;
wherein the tubular section doubles a as a handle for allowing a user to grasp and manipulate the applicator head;
means on both the applicator head and the container for securing the applicator head to the container and, at an appropriate time, to manipulate the applicator head in a specific manner in order to open the barrier, thereby releasing the liquid solution into the tubular section and allowing the liquid solution to be absorbed by the pad.
